(19) United States
(12) Patent Application Publication
(10) Pub. No.: US 2013/0123319 A1
(43) Pub. Date: May 16, 2013

(54) Medthod of Treating a systemic inflammatory disorder and damaged internal tissues

(71) Applicant: Thomas Benedict Bryan, Merced, CA (US)

(72) Inventor: Thomas Benedict Bryan, Merced, CA (US)

(21) Appl. No.: 13/373,445

(22) Filed: Nov. 15, 2011

(51) Int. Cl. A61K 31/047; A61P 17/16; A61P 29/00; A61K 31/4172; A61P 37/00

(52) U.S. Cl. 514/400; 514/738

(57) ABSTRACT

3% Glycerin plus 3% amino acids intravenously used for nutritional purposes produces a rapid remission of an acute exacerbation in Behcet's Disease. External manifestations such as oral, vulva, penile and skin lesions rapidly heal when Glycerin is added to the amino acids. While the disease process in Behcet's Disease is likely improved by amino acids alone, the robustness of the healing of open lesions and the overall well-being of the patient in acute exacerbations is greatly enhanced by the addition of 3% Glycerin to the IV fluid. IV 3% Glycerin and 3% Amino Acids also produced remissions in Myasthenia Gravis, Guillain-Barre Disease, and Chronic Inflammatory Demyelinating Polyneuropathy. 3% Glycerin with amino acids could cause healing in many autoimmune diseases and can help external and internal lesions to heal.

Glycerin also prevents bacteria from forming a biofilm which in most infections is necessary for the bacteria to become pathologic.

### FIELD OF INVENTION

The present invention pertains to the treatment of systemic inflammatory disorders such as autoimmune disorders and promotes healing of internally damaged tissues in the body. It also should help in the treatment of Bacterial Disease and the healing of tissue lesions or damage to these tissues.

### BACKGROUND

In 1990 I began to treat a patient with Myasthenia Gravis without Thymoma, an autoimmune disorder, which causes a decrease in the availability of Acetylcholine at the muscle endplate. Pyridostigmine, which increases the amount of Acetylcholine available at the endplate, and later Prednisone, was added for control of her myasthenia. The later acts by reducing the inflammatory response at the muscle endplate. In 1992 she underwent a bowel operation for correction of an enterorectal fistula and was unable to eat orally or gastrically to be fed. She was put on the standardized intravenous nutritional protocol in the hospital in which I worked, which included 3% Glycerin plus 3% Amino Acids, (see Table 1) at a rate of 100 ccs/hour. This was given for 6 days. There was no change in her Myasthenia Gravis. Twelve days later she was readmitted with abdominal pain and the same IV nutrition was administered. On day 5 of the hospitalization she developed a cholinergic crisis, which was relieved by Atropine. Pyridostigmine and Prednisone were discontinued. The patient continued to feel stronger in subsequent days. Afterwards, she was discharged home and was given a combination of 3% amino acids and 3% glycerin along with vitamins and minerals plus lipids, as in Table 1, over a period of 3 months. During this time she did not need Pyridostigmine or Prednisone, despite continued elevation of the Acetylcholine receptor antibody titer. 8 days after discontinuation of the intravenous feeding she began to develop symptoms of Myasthenia Gravis again and was put back on Pyridostigmine and 6 months later had to have Prednisone added. Three and a half months later she was admitted to the hospital because of an abdominal pain, dehydration, and difficulty swallowing and at this time was given Freamine (B. Braun, Table 2) which included a stronger formulation of amino acids without glycerin, see Table 2. She agreed to this change in her IV infusion to see if it made a difference. She again went into remission. Her Myasthenia remained in remission for 6 months at which time she was put back on Pyridostigmine. 5 months later Prednisone was added. She was readmitted to the hospital 4 years later because of difficulty swallowing and again was given intravenous 3% Glycerin and 3% Amino Acids. She inadvertently was not prescribed Pyridostigmine and Prednisone and was discharged home 18 hours later. She again went into remission and remained in remission until her death 4 years later.

In 1997 a patient presented with Guillain-Barre disease, which was most severe in her pelvic musculature. She was informed that the accepted treatment for this was intravenous immunoglobulin (IVIG). After being told about the possible side effects of IVIG and the success with treating the Myasthenia Gravis with an infusion of amino acids and amino acids with glycerin she opted for the mixture of 3% Amino Acids with 3% Glycerin IV. She had a very rapid recovery going from paralysis of her legs and weakness of her shoulders to being able to walk and discharged home on the 5th day of the infusion. She continued having some mild weakness of her legs indefinitely after that. In late 2009 a patient developed a mild to moderate form of Guillain-Barre Syndrome after receiving an H1N1 flu shot. Again, she opted for the amino acid 3% and glycerin 3% infusion and had a rapid improvement over the next few days.

In January 2002 a 49 female presented with Chronic Inflammatory Demyelinating Polyneuropathy (CIDP) with weakness and numbness of the legs and a loss of deep tendon reflexes. She improved after being on Methylprednisolone 100 mg IV daily for 3 weeks. In April 2002 she was readmitted with a recurrence of her symptoms, which responded much more rapidly on this occasion with a combination of Methylprednisolone and intravenous 3% amino acids and 3% glycerin, (Table 1). Also exacerbations of her CIDP were prevented by an infusion of 2 liters of 3% amino acids and 3% glycerin (Table 1,) on a weekly basis while at home. When this was reduced to a monthly treatment exacerbations of CIDP reoccurred. From September 2003 to March 2005 the patient was free of CIDP. Later she responded to infusions of both 3% amino acids with 3% glycerin and also to IVIG.

All of the above treated diseases are thought to be due to an autoimmune disease.

### BEHCET'S SYNDROME (DISEASE)

Behcet's Syndrome is a worldwide disorder that seems to be more severe and more common along the Silk Route and in Japan. In the West it is thought to be milder and self-limiting and more rare though this is debatable, as I myself have seen over 20 patients with Behcet's Disease since the year 2000. Exacerbations usually responded to high doses of Prednisone.

Eleven of these patients whose exacerbation of Behcet's failed to respond to high dose Prednisone responded rapidly to an infusion of 3% amino acids and 3% glycerin with healing of the lesions in their mouth and pharynx, on their genitals, buttocks and skin. They also felt much improved. These infusions lasted between 2 days and 5 days before a good response was obtained.

Recently a female patient with an exacerbation of Behcet's was given a prescription for a 5 day infusion of 3% amino acids and 3% glycerin to be infused at home. She failed to respond to the infusion. This prompted me to call the home infusion company and I discovered that she was given an infusion of amino acids which were quite similar to the amino acids in Table 1 but without the glycerin. Beginning about 2 weeks after the infusion, the mouth ulcers which she was having for 2 years cleared up as did the myalgia. She still suffers a lot of stress due to pain in her knees and shoulders and is scheduled to have these replaced. Also one of the 11 patients who responded favorably in the past to the 3% amino acids and 3% glycerin within a period of 48 hours in the hospital failed to respond to home infusion on 2 occasions and I can only presume that he was not given the glycerin on either of those occasions. This happened back in the year 2000. He has been lost to follow-up.

In 2004 a patient had been in the hospital for 3 weeks while receiving antibiotics for a large skin ulcer on her leg. She had been treated with numerous antibiotics without success. I suggested she be tried on an infusion of 3% Glycerin and 3% Amino Acids and the next day one could see healing occurring. She was kept on the infusion for 72 hours and discharged home. One week later the ulcer, which measured 1 inch by ½ inch, had completely healed. Later on she presented with signs typical of Behcet's Disease.

While many of these disorders can be treated with intravenous immunoglobulin (IVIG), IVIG is very expensive costing thousands of dollars per infusion whereas the intravenous combination of 3% amino acids and 3% glycerin is relatively inexpensive. There is also significant morbidity with IVIG including cardiac failure, myocardial infarction, acute tubular necrosis of the kidneys, aseptic meningitis and a reversible encephalopathy. One can also develop anaphylaxis, hemolytic anemia and immune complex arthritis among many others. It is also possible that a viral or prion disease may be transferred from a blood donor which is necessary to make IVIG.

Intravenous use of 3% amino acids plus 3% glycerin for nutritional purposes has been used in the United States since 1976 without any significant morbidity.

Glycerin is a simple polyol compound. It is a colorless, odorless, viscous liquid that is widely used in pharmaceutical formulations. Glycerin is the backbone of all lipids known as triglycerides. Glycerin is sweet tasting and of low toxicity. It is produced primarily by saponification of fats as a byproduct of soap making. It is used in the manufacture of shortenings and margarines. It is characterized by the American Dietetic Association as a carbohydrate. It has a lower glycemic index than sugar and some dietary advocates accept glycerin as a sweetener compatible with low carbohydrate diets. It is used in medical, pharmaceutical and personal care preparations, mainly as a means of improving smoothness, providing lubrication and as humectants. It is found in cough syrups, expectorants, tooth paste, mouth washes, skin care products and shaving cream. In its solid form glycerin is used as a tablet holding agent. Glycerin soap is used for people with sensitive, easily irritated skin and it prevents skin dryness. (Google search)

### GLYCERIN IN THE TREATMENT OF INFECTIONS

In the patient with the ulcer on her leg, antibiotics aimed at cultures from the skin lesion even in the presence of topical antiseptics failed to stop the progression of the infected skin. A course of 3% Glycerin and 3% Amino Acids intravenously quickly reversed the course of the disease. Despite having skin that prevents invasion of bacteria into our bodies, bacteria do enter as single units and are quickly engulfed by our macrophages.

However, since Costerton in 1978 we have begun to realize that bacteria have to first organize themselves into “biofilms” in order to become pathogenic. 80% of human infectious diseases are biofilm based infections. (Minutes of the National Advisory Dentistry and Craniofacial Research Council—153^{rd} Meeting, 1997)

Dentists have known for many years about the usefulness of Glycerin externally for the dissipation of biofilms such as halitosis and periodontal disease. Likewise, it has been used by physicians and nurses for Psoriasis, burns, bites, cuts, rashes and bedsores. The problem with both systemic and topical antibiotics is that they cannot be used on wound healing without adjunct topical antiseptics and anti-biofilm strategies (Evidence of Biofilms in Wounds and the Potential Ramifications, D. D. Rhoads, R. W. Wolcott, K F Cutting and S. L. Percivals) In 1905 J. J. Kinyoun, MD, PhD of Philadelphia while working to preserve viruses had reduced bacteria in the specimens through the use of Glyercin especially when mixed with serum and that the addition of both were more potent than using either of them alone. (Journal of Experimental Medicine, Vol. 7(6); Nov. 25, 1905)

It seems that glycerin, especially when mixed with blood serum is a potent antibacterial that seems to breakdown pathogenic bacterial biofilm and that the intravenous use of sterile glycerin which would be mixed with the patient's serum would significantly aid in the infections caused by biofilm.

Biofilms can be up to one thousand times more resistant to antibiotics than to individual bacteria, according to Garza, Syracuse University, who had worked with scientists from Miami University, Madison, Wis., and Stanford University. (Science News, Jun. 24, 2011, Pages 22-35)

This approach has not yet been tried. Other biofilm preventatives such as furantoins are toxic whereas Sterilized Glycerin is non-toxic.

### SUMMARY OF INVENTION

Through my review and observations in these cases I discovered that the administration of intravenous glycerin has a potent beneficial effect in the treatment of autoimmune diseases, especially noticeable in Behcet's Disease and produces a quick or rapid improvement whereas intravenous amino acids without the glycerin seems to cause improvement but at a much slower rate.

IV Glycerin speeds healing of damaged tissues such as lesions in the mouth, genitals and skin and on one occasion improved central nervous system involvement rapidly in one patient with neuro-Behcet's disease.

I found that IV Glycerin can aid in the treatment of an infectious ulcer and it is known to breakdown bacterial biofilm.

TABLE 1
Individual elements and the Quantity of each Element
in 100 cc of a First Standardized Parenteral Nutritional Protocol
Formulation of Procalamine (B. Braun prior-by McGaw)

| Elements | Amount | |
|---|---|---|
| Distilled Water | | |
| 3% Glycerin | | |
| Essential Amino Acids | | |
| Isoleucine USP | 0.21 | gms |
| Leucine USP | 0.27 | gms |
| Lysine as Lysine acetate USP | 0.31 | gms |
| Methionine USP | 0.16 | gms |
| Phenylalanine USP | 0.17 | gms |
| Threonine USP | 0.046 | gms |
| Valine USP | 0.2 | gms |
| Non-Essential Amino Acids | | |
| Alanine USP | 0.21 | gms |
| Arginine USP | 0.29 | gms |
| Histidine UAP | 0.085 | gms |
| Proline USP | 0.34 | gms |
| Serine USP | 0.18 | gms |
| Glycine USP | 0.42 | gms |
| Cystein USP | 0.014 | gms |
| Lipids | 10 | gm |
| Soybean Oil | * | |
| Egg Phosphatides | * | |
| Electrolytes | | |
| Sodium Chloride | 80 | mEq/liter |
| Sodium Phosphate | 25 | mEq/liter |
| Potassium Chloride | 30 | mEq/liter |
| Calcium Gluconate | 12 | mEq/liter |
| Magnesium Sulfate | 8 | mEq/liter |
| Zinc Sulfate | 20 | mEq/liter |
| Trace Elements | | |
| Zinc | 5 | mg |
| Copper | 2 | mg |
| Manganese | 0.5 | mg |
| Chromium | 10 | mcg |
| Selenium | 60 | mcg |
| Daily Vitamins | | |
| Ascorbic Acid | 500 | mg |
| Vitamin A | 1000 | i.u. |
| Vitamin D | 1000 | i.u. |
| Thiamine | 50 | mg |
| Riboflavine | 10 | mg |
| Pyridoxine | 5 | mg |
| Niacin | 100 | mg |
| Vitamin E | 5 | i.u. |
| Dexpanthenol | 25 | mg |
| Vitamin K | 10 | mg |

TABLE 2
Individual Elements and the Quantity of Element in
100 cc of the Second Standardized Parenteral Nutritional Protocol
Formulation of Freamine (B. Braun)

| Elements | Amount | |
|---|---|---|
| Distilled Water | | |
| Essential Amino Acids | | |
| Isoleucine USP | 0.59 | gms |
| Leucine USP | 0.77 | gms |
| Lysine as Lysine acetate USP | 0.87 | gms |
| Methionine USP | 0.45 | gms |
| Phenylalanine USP | 0.48 | gms |
| Threonine USP | 0.34 | gms |
| Tryptophan USP | 0.13 | gms |
| Valine USP | 0.56 | gms |
| Non-Essential Amino Acids | | |
| Alanine USP | 0.60 | gms |
| Arginine USP | 0.81 | gms |
| Histidine UAP | 0.24 | gms |
| Proline USP | 0.95 | gms |
| Serine USP | 0.56 | gms |
| Glycine USP | 1.19 | gms |
| Cystein USP | 0.014 | gms |
| Glycerin as Glycerol | 3 | gms |
| Electrolytes | | |
| Calcium Glucanate | 9.2 | mEq/liter |
| Sodium Chloride | 45.0 | mEq/liter |
| Potassium Acetate | 8.1 | mEq/liter |
| Calcium Gluconate | 12 | mEq/liter |
| Magnesium Sulfate | 8.1 | mEq/liter |
| Potassium Phosphate | 10.0 | mmoles/liter |
| Sodium | 10.1 | mEq/liter |
| Phosphate | 20.0 | mmoles/liter |
| Acetate | 72.0 | mEq/liter |
| Chloride | 3.0 | mEq/liter |
| Multi-Vitamins (Daily Amounts) | | |
| Ascorbic Acid | 100 | mg |
| Vitamin A | 1 | mg |
| Vitamin D | 5 | mcg |
| Thiamine | 3 | mg |
| Riboflavin | 38 | mg |
| Pyridoxine | 4 | mg |
| Niacin | 40 | mg |
| Vitamin E | 10 | mg |
| Dexpanthenol | 15 | mg |
| Biotin | 60 | mcg |
| Folic Acid | 400 | mcg |
| Cyanocobalamin | 5 | mcg |

What is claimed:

1. A method of treating an autoimmune disease in a patient: the method comprising the intravenous infusion of glycerin or the combination of intravenous glycerin and intravenous amino acids.

2. A method for treating systemic inflammatory disorders through the intravenous infusion of glycerin or glycerin plus amino acids.

3. A method of speeding healing of damaged tissues or exposure to radiation through the intravenous infusion of glycerin or glycerin plus amino acids.

4. A method to intercept bacterial biofilms and prevent infections through the intravenous use of Glycerin.
